(19) United States
(12) Patent Application Publication
(10) Pub. No.: US 2011/0048432 A1
(43) Pub. Date: Mar. 3, 2011

(54) U-wraped examination drape

(76) Inventor: Edna M. Hutchinson, San Jacinto, CA (US)

(21) Appl. No.: 12/583,931

(22) Filed: Aug. 28, 2009

(51) Int. Cl. A61B 19/08; A61B 19/12

(52) U.S. Cl. 128/853; 128/856

(57) ABSTRACT

An new Improved Medical Examination Drape Designed in the shape of a pair of pants, a garment used in Hospital's, Clinics, and Doctors office's, to provide privacy, and coverage over the female patients entire lower body portion from the waistband down covering, the legs, hips, thighs, and stomach, has a attached lower front portion a hidden middle center slit opening between the upper inner thighs extending back to the rear drawstring waistband tie opening making it easier, faster, and better, for Doctors to perform pap smear's and pelvic examination without exposing the lower body portion.

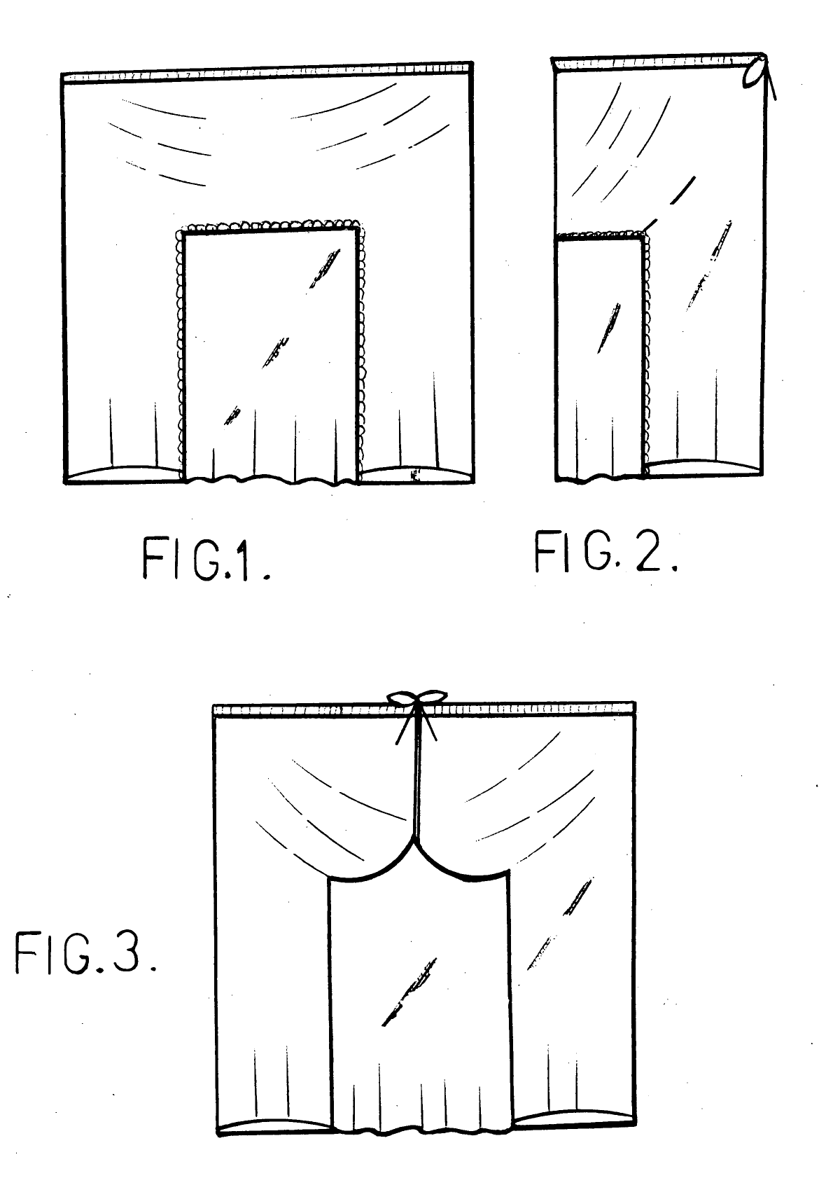

FIG. 1 is a front elevational view of a U-Wrapped Examination Drape Showing by new design.

FIG. 2. is a left side Elevational view of a U-Wrapped Examination drape.

FIG. 3. is a rear Elevational View of a U-Wrapped Examination Drape showing a rear opening and tie.

### SPECIFICATION

The U-Wrapped Examination Drape is used for female patents in a Hospital, Doctor's office, and Medical Clinic's for privacy, comfort, and coverage over the lower body portion when having a pap smear or a pelvic examination done, on themselves to keep from being exposed, during many examination, made a one piece pants shape garment easily to slip on and off covering over the lower body portion without exposing the legs, hips, thighs, and stomach used for coverage comfort, and privacy made a middle center slit opening between the inner upper thighs extending to rear waistband tie opening used for easier and better access for examination, without exposing the lower body portion.

### BACKGROUND OF THE INVENTION

After working in Hospital's, Doctors office's, and Medical Clinic's, for many years I found a great need for a Medical Examination Drape that keep female patients lower body portion covered without being exposed when having a pelvic examination.

### BRIEF SUMMARY OF THE INVENTION

The advantages of the U-Wrapped Examination Drape is it keep female patients covered during pap smears, and pelvic examinations, designed a one piece easily slip on and off pants-shaped examination drape garment that cover the lower body portion from the waistband down for privacy, comfort, and coverage, without exposing the lower body portion when having a medical pap smear and pelvic examination done on themselves.

The Prior Examination Draped is a folded bed sheet and a disposable paper sheet used to cover the lower body portion during a pelvic examination which sometime slide off the body or slide to the side exposing the lower body portion during the pelvic examination.

1. The ornamental design for a U-Wrapped Examination Drape as shown and described, used for females patients in Hospital's, Clinic's, and Doctor's, office's, for coverage over the lower body portion during Medical Pelvic Examination or pap smear's.

2. The ornamental design for a U-Wrapped Examination Drape as shown and described, designed a one piece pants shaped slip on easily examination drape garment used for female patients in a medical setting for privacy and coverage over the lower body portion without exposing the hips, legs and thighs, and stomach when having a pelvic examination, done on themselves has hidden front to back middle center slit opening between the upper inner thighs extending back to the rear drawstring waistband tie, opening used for easier and better access for pap smear and pelvic examination.

3. The ornamental design for a U-Wrapped Examination Drape as shown and described worn my female patients in a hospital, Doctor office, or clinic, a one piece stylish pants shape examination drape garment used for coverage over the full lower body portion when having a pap smear or pelvic examination, made a hidden middle center slit opening between the inner upper thigh full length extending from front to rear drawstring waistband tie opening for easier pelvic examination without exposing the lower body portion.
